(19) United States
(12) Patent Application Publication
(10) Pub. No.: US 2012/0017897 A1
(43) Pub. Date: Jan. 26, 2012

(54) Tracheal Tube Cuff Pressure Indicator

(76) Inventors: Sridhar Ranganathan, Suwanee, GA (US); Andrew Baker, Norcross, GA (US); Shawn R. Feaster, Johns Creek, GA (US); Edward B. Madsen, Cumming, GA (US); Phillip Schorr, Cumming, GA (US)

(21) Appl. No.: 12/839,521

(22) Filed: Jul. 20, 2010

(51) Int. Cl. A61M 16/04

(52) U.S. Cl. 128/202.22

(57) ABSTRACT

A pressure indicator for cuffed tracheal tubes is provided. This indicator uses an indicator balloon on the proximal end of the inflation lumen and in fluid communication with the tracheal tube cuff. As the pressure in the cuff changes, the balloon will inflate and deflate. An indicator needle rests on the balloon and moves in response to the inflation and deflation of the balloon, thus showing the state of inflation of the cuff.

The present disclosure relates to a tracheal catheter or tube used for mechanical ventilation of a hospital patient, by insertion of the tube into the trachea of the patient. In particular, the present disclosure relates to a tracheal tube having means to indicate the pressure in the balloon or cuff that occludes the trachea and that is located on the tube.

There are two principal types of tracheal catheters or tubes; the endotracheal tube (ET tube) and the tracheostomy tube (trach tube). The ET tube is inserted through the mouth of a patient and guided past the vocal cords and glottis into the trachea. The trach tube is inserted directly into the trachea through a stoma created in the throat and the tracheal wall by surgical means and enters the trachea below the glottis. Both types of tube have a relatively large main ventilating lumen that delivers the air from a mechanical ventilating device to the lungs. Both types of tubes typically terminate at a position above the carina, anterior to a position between the second and fourth thoracic vertebrae. Gases may then be introduced through the tracheal tube and into the lungs of the patient.

The primary purposes of tracheal intubation are to mechanically ventilate the patient's lungs, when a disease prevents the patient from normal, breathing induced ventilation, and to apply anesthetic gases during surgical intervention. In order to create enough air pressure to accomplish such mechanical ventilation and to prevent escape of gases past the tube, it is necessary to seal the passageway around the tracheal tube. A seal may be produced by the use of an inflatable cuff or balloon formed integrally with and surrounding the tracheal tube. When the tracheal tube has been introduced into the patient's trachea, the inflatable cuff will normally be located a few centimeters above the carina and within the tube-like trachea

The inflatable cuff is then inflated so as to engage the wall of the trachea and thereby seal the trachea and prevent gases being introduced through the tracheal tube from simply turning back up around the tube and passing out of the patients mouth and nose.

The proper inflation of the cuff is quite important. Under-inflation can allow secretions which would normally be directed away from the trachea and into the digestive system to instead follow the path of the ET tube and flow around the inflatable cuff of the tracheal tube downward into the lungs. These contaminated secretions may result in the patient developing ventilator acquired pneumonia or VAP, a major problem in modern medical treatment. Under inflation can also be a cause of poor air sealing allowing air to leak upwardly past the cuff, reducing the effectiveness of the ventilator. Under-inflation of the cuff is, however, less common than over-inflation.

Over-inflation of the inflatable cuff is more common and can result in compression of the tissue in the wall of the trachea, possibly resulting in stenosis. If the cuff on the trachael tube is inflated to a pressure greater than about 40 cmH₂O , the capillary perfusion pressure of the trachea is exceeded. It is then possible to develop mucosal ischemia, chondritis, granulation tissue and, finally, scar and contraction of scar and fibrosis tissue, which leads to the stenosis. High-volume low-pressure cuffs have a much lower rate of tracheal stenosis than the low-volume high pressure cuffs used previously, but any low-pressure cuff can be easily converted to a high pressure cuff by over-inflation.

Current methods of determining inflatable cuff pressure are quite haphazard. A common method is for the medical professional to feel the pressure of the pilot balloon, located on the proximal end of the tube, between his forefinger and thumb. Considering the thickness of the pilot balloon and the limitation of the human hand to feel such subtle differences in pressure, this is of relatively limited utility. Some physicians count the number of times they pump the inflation device for the cuff, though the differences in size of the trachea from patient to patient makes this method unreliable at producing a safe pressure as well.

Current methods also include devices that attach to the inflation line of the cuff and provide information related to the cuff pressure. These have varying forms that range from fairly complex dial gages to more simple bellows or spring/rolling diaphragm type configurations. These devices tend to be on the bulky side such that they are not amenable to be readily used for continuous cuff pressure monitoring. Moreover, these approaches require direct access to the inflation line thus creating a new potential failure mode for the cuff to deflate if there is an accidental leak in the connections.

Use of the pilot balloon as a cuff pressure estimation site has also been identified previously. Clip-on pressure gages that provide either a numerical value or a qualitative estimate of the pressure have been discussed. However, the challenge and potential error introduced by the pilot balloon material on the accuracy and sensitivity of cuff pressure estimation has not been recognized previously.

What is needed is a tracheal catheter having an indicator of pressure so that the user knows how high the pressure is in the cuff, or at least that the pressure in the cuff does not exceed safe limits.

### SUMMARY

The present disclosure improves upon a cuffed tracheal catheter by providing an indicator for the pressure in the cuff. The pressure indicator may be attached to the proximal end of the cuff inflation lumen. The pressure indicator uses an indicator balloon that inflates and deflates in response to the pressure in the inflatable cuff. The balloon as described here may have walls that may or may not stretch. The walls of the balloon may be heterogeneous in the sense that some parts of it may be rigid while others flexible. The balloon is desirably in fluid communication with the tracheal tube cuff. An indicator needle is desirably in mechanical communication with the balloon and moves in response to the inflation and deflation of the balloon, thus showing the state of inflation of the cuff.

Other objects, advantages and applications of the present disclosure will be made clear by the following detailed description of a preferred embodiment of the disclosure and the accompanying drawings wherein reference numerals refer to like or equivalent structures.

### BRIEF DESCRIPTION OF THE DRAWINGS

FIG. 1 is a view of an ET tube of the prior art.

FIG. 2 shows a design of a pressure indicator that may be used with a tracheal tube.

FIG. 3 shows another embodiment of an indicator pursuant in which the needle is perpendicular to the inflation line.

FIG. 4 shows a version of a pressure indicator that may be retrofitted onto the pilot balloon of an older style ET tube.

FIG. 5 is a graph of needle displacement sensitivity (Y axis) versus to cuff pressure (X axis).

### DETAILED DESCRIPTION

Reference will now be made to the drawings in which the various elements of the present disclosure will be given numeral designations and in which the disclosure will be discussed so as to enable one skilled in the art to make and use the disclosure. It is to be understood that the following description is only exemplary of the principles of the present disclosure, and should not be viewed as narrowing the pending claims. In particular, though most references herein are to an ET tube since the problem of cuff overpressure is greater when using ET tubes, these teachings apply equally to trach tubes. Those skilled in the art will appreciate that aspects of the various embodiments discussed may be interchanged and modified without departing from the scope and spirit of the disclosure.

In manufacturing a tracheal tube, the main cannula is generally extruded by conventional means. As it is extruded in a never ending tube, the cannula is generally given three lumens; the main respiratory lumen, a cuff inflation lumen, and a suction lumen, separated by internal walls. There may be more lumens extruded into the cannula for additional functions and the suction lumen is sometimes dispensed with, but the three recited are the most common. These lumens extruded into the cannula extend the entire length of the cannula. Once the cannula is cut to the proper length, the cuff inflation port and the suction port are located and “skived” or cut out, a technique that is well known to those skilled in the art. This allows liquid communication of each lumen (suction and inflation) through the wall of the cannula to its respective port, opening into the space outside the cannula. The remaining distal portion of the cuff inflation and suction lumens are then blocked below the skived port, generally with a sealing plug. The respiratory lumen extends the entire length of the cannula and is not skived out. The cuff is then attached to the cannula, usually adhesively, in a location over the inflation port.

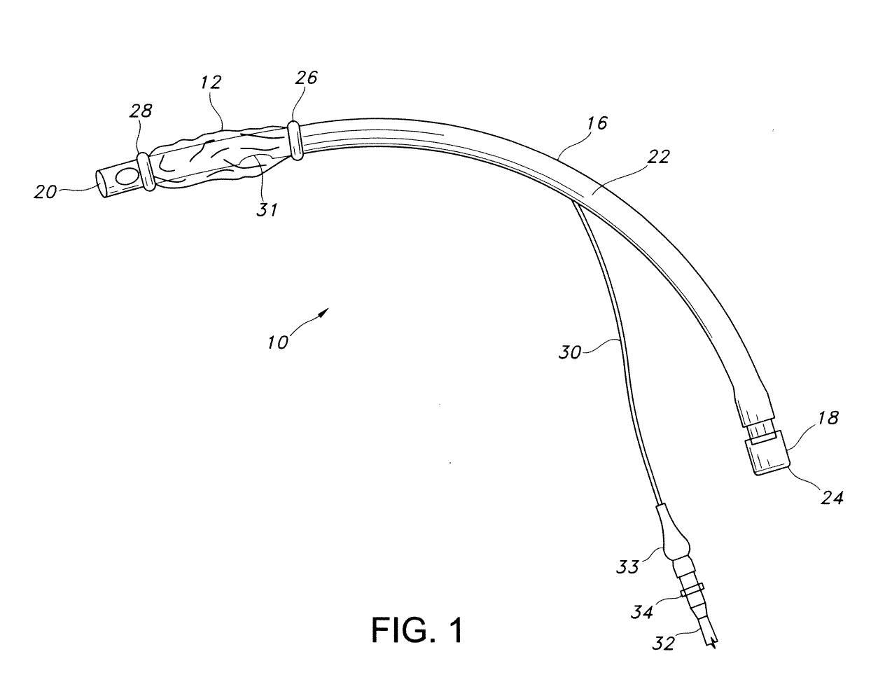

Turning to the drawings, FIG. 1 illustrates a prior art ET tube 10 including an inflatable cuff 12. Tube 10 includes a cannula 16 having an open proximal end 18 and an open distal end 20. The cannula 16 defines a gas-conveying lumen 22 for mechanical ventilation of a patient. The proximal end 18 usually includes a connector 24 configured for attachment to a mechanical ventilator (not shown). The inflatable cuff 12 is mounted on the cannula 16 adjacent the distal end 20 of the cannula 16, covering the skived out inflation lumen port(s) 31. The cuff 12 is mounted on the cannula 16 by one or more collars. In FIG. 1, cuff 12 may be mounted on cannula 16 by a first or proximal collar 26 and a second or distal collar 28. The proximal end of the inflation lumen 30 connects to an inflation line 32 and a pilot balloon 33 that are used in the process of inflating the cuff 12.

During the insertion of the tube 10, the cuff 12 is at least partially collapsed. Once properly in place, the cuff 12 may be inflated via the inflation lumen 30 and cuff inflation port(s) 31 formed in or otherwise associated with the cannula 16. The inflation lumen 30 may be coupled to an inflation line 32 with the pilot balloon 33 and terminating at its proximal end in a fitting 34 that allows inflation of the cuff 12 via the inflation lumen 30 and cuff inflation port(s) 31. The cuff 12, inflation lumen 30, inflation line 32 and pilot balloon 33 are thus in fluid communication.

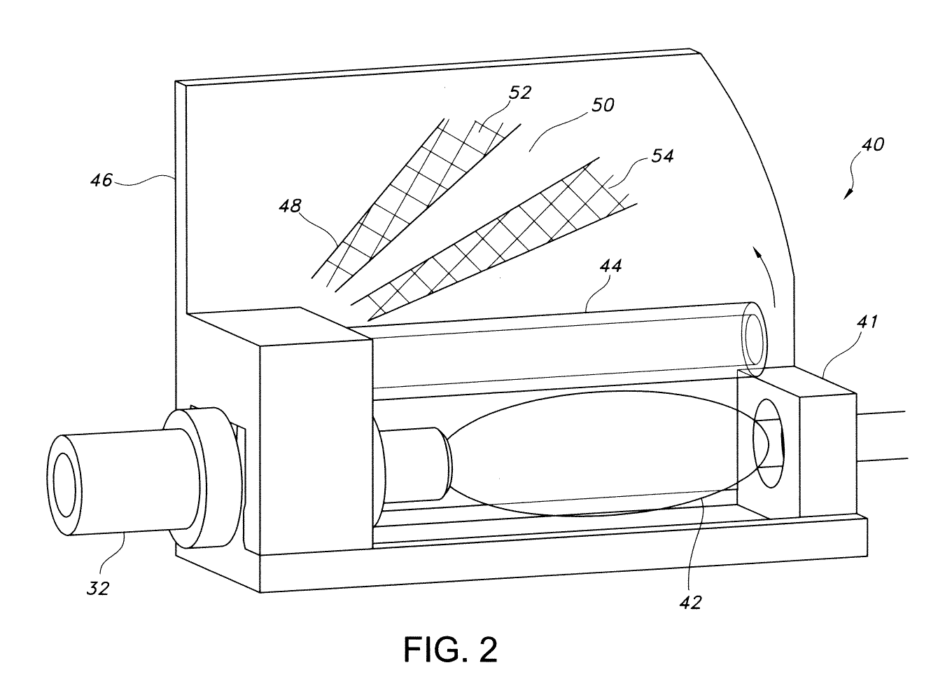

FIG. 2 shows a design of a pressure indicator 40 that may be used with a tracheal tube 10 of FIG. 1 to give a better indication to a medical professional that the cuff 12 is properly inflated. The pressure indicator 40 may be placed at a point in the inflation line 32 or may replace the pilot balloon 33 if desired. The indicator 40 shown in FIG. 2 has been placed in the inflation line 32 and the balloon 42 is in fluid communication with the cuff via the inflation lumen as described above. The indicator 40 has a frame 41 with the balloon 42 and a needle 44 that is in mechanical communication with the balloon 42. In this case the needle 44 rests on the balloon 42 and responds to the inflation level of the balloon 42 by moving upwardly (the direction of the arrow in FIG. 2) to indicate greater inflation or expansion of the balloon 42 and downwardly to indicate lesser inflation or contraction of the balloon 42. The pressure indicator 40 may optionally have a backing 46 which may have a scale showing the absolute or relative level of inflation. The scale 48 may, for example, have red and green zones to indicate bad and good pressure ranges, respectively. In FIG. 2, the scale 48 may have three ranges as shown, with the center range 50 colored green and the other two ranges 52, 54 colored red. Alternatively, a scale 48 having actual balloon pressures may be used.

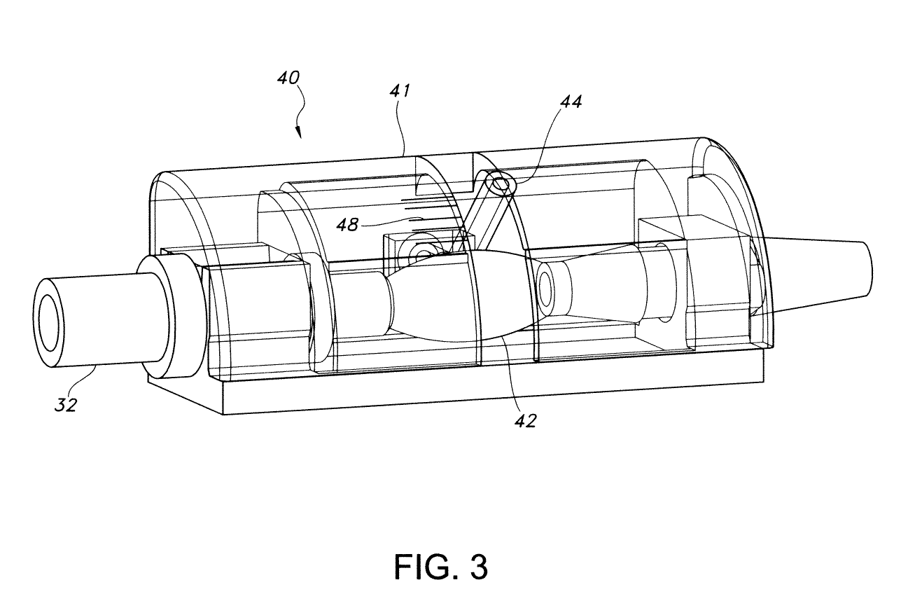

While the balloon 42 shown in FIG. 2 may have a shape as shown, the balloon may be of any other functional shape. The balloon may have a well defined shape in its inflated state but it should be recognized that in the deflated state it may not. Other inflated shapes, in fact, may enhance the sensitivity of the balloon 42. Spherical, toroidal, and cylindrical, oblate or prolate spheroids and other shapes may be chosen based on routine experimentation, within the ability of the skilled person. The illustration of an oblong balloon is not meant to limit the scope of this disclosure. FIG. 3 shows another embodiment of an indicator 40 pursuant to these teachings. In this case, the indicator needle 44 is perpendicular to the inflation line 32 and again is in mechanical communication with the balloon; e. g. in direct contact with (resting on) the balloon. It is believed that this arrangement provides a more sensitive reading of the pressure in the balloon 42 and so is more responsive to changes in cuff pressure. A scale 48 may be placed on the frame 41. Again, the shape of the balloon 42 may be optimized through routine experimentation to provide a still more sensitive response.

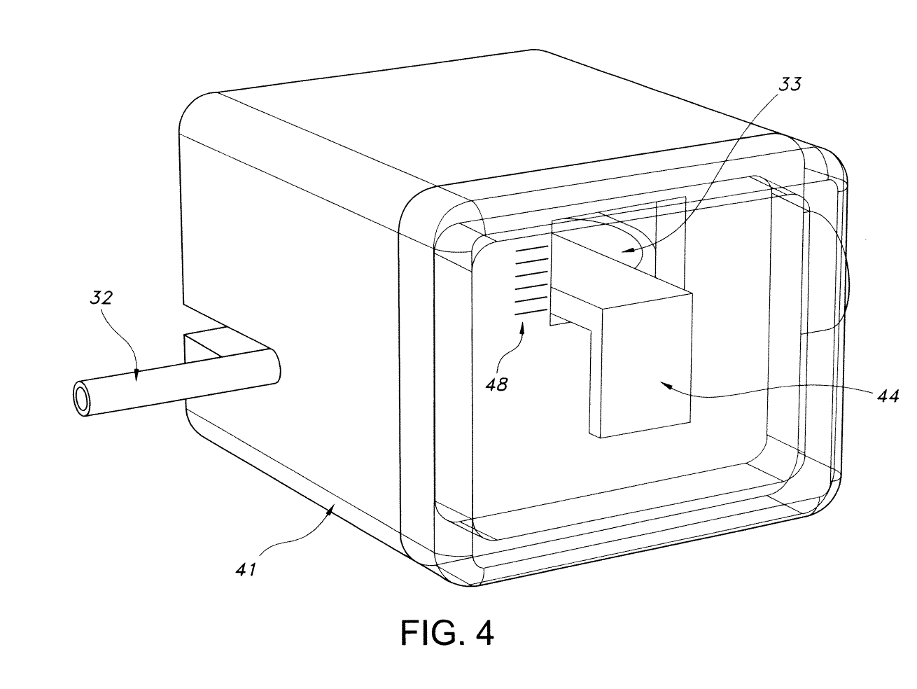

FIG. 4 shows a version that may be retrofitted onto the pilot balloon 33 of an older style ET tube. The pilot balloon serves as the indicator balloon in this embodiment. The frame 41 fits over the pilot balloon 33 with the indicator needle 44 resting on the pilot balloon 33. As the pilot balloon 33 inflates, the needle 44 will move, thus indicating the state of inflation of the cuff. A scale 48 may be printed on the exterior surface of the frame 41 to indicate relative or absolute level of inflation, as in the previous embodiments.

The indicator needle 44 is desirably constructed of an elastic polymer with little or no plastic deformation or creep over the range of strains that occur over the relevant pressure range and time scales of use. One end of the needle 44 may be attached to the frame 41 and the other end is unattached. The unattached end of the needle 44 may be inflexible and colored or shaped to provide visual clarity for the indicator portion of the needle. The attached end is either hinged or held fixed directly to the frame 41. If the attached end is hinged, the needle is preferably rigid while if the attached end is fixed, the needle is preferably flexible. This will ensure one degree of freedom in deflection of the needle tip that can then be robustly translated into a pressure value.

If the indictor is attached to the frame through the use of a hinge, a restorative force must be provided by, for example, a spring, weight or other means known in the art, to move the needle towards its starting point (e.g. downward) as the pressure in the balloon decreases.

If the attached end is fixed directly to the frame without a hinge, the proper choice of materials will provide a restorative force so that the needle 44 will bend upwardly in response to increased pressure (and therefore size) of the balloon 42 and will return to a lower position should the balloon 42 pressure decrease. In another embodiment, as the indicator balloon inflates it pushes against a fixed support structure that holds the indicator needle as well.

The indicator balloon 42 is desirably made from a thin and compliant material so that it can respond quickly to changes in the cuff pressure and be minimally isolating between the internal pressure and ambient. Exemplary materials include soft, pliable polymers such as polyethylene teraphthalate (PET), low-density polyethylene (LDPE), polyvinyl chloride (PVC), polyurethane (PU), polyolefin or polydimethylsiloxane (PDMS) polymers. The balloon 42 should be very thin; with a thickness on the order of 25 microns or less, e.g. 20 microns, 15 microns, 10 microns or even as low as 5 microns in thickness, though at least 1 micron. The balloon should be able to indicate changes in the system pressure at quite low pressures, as a low pressure cuff desirably operates at an inflation pressure of about 25 to 30 cmH₂O or less. Very thin balloons, for examples those described in U.S. Pat. Nos. 6,802,317 and 6,526,977, can successfully operate at even lower pressures, such as 20 cmH₂O, 15 cmH₂O or even less. An appropriate range for an indicator balloon would be, for example, from 0 to 70 cmH₂O or more desirably from 15 to 45 cmH₂O.

The inflation of the indicator balloon has two phases. The balloons are manufactured to have a specified shape when the balloon material is unstretched. In a blow mold process for example a tube of the given raw material is placed into a cavity mold that has the designed balloon shape. The tube is heated above its softening point and inflated. The balloon mold is cooled and the newly formed balloon material solidifies into this designed shape (with some small changes as it cools). The first phase of the inflation of the balloon occurs as the incoming air re-inflates the balloon to this original shape. In this phase the balloon material undergoes very little if any stretching while it inflates. In this phase the air pressure is reshaping, unfurling or “filling out” the balloon only and not stretching the wall of the balloon. The second phase occurs after the balloon reaches the initial balloon shape. Adding pressure to the balloon after it has reached this shape will cause additional change in shape but will also cause the walls of the balloon to stretch. A pressure indicator can be constructed to make use of balloon deformation in either phase of the inflation. The non-linear behavior of the second phase inflation and the complexities of the shape/pressure behavior at the transition from one phase to the other results in desirably using the first phase in the practice of the disclosed pressure indicator.

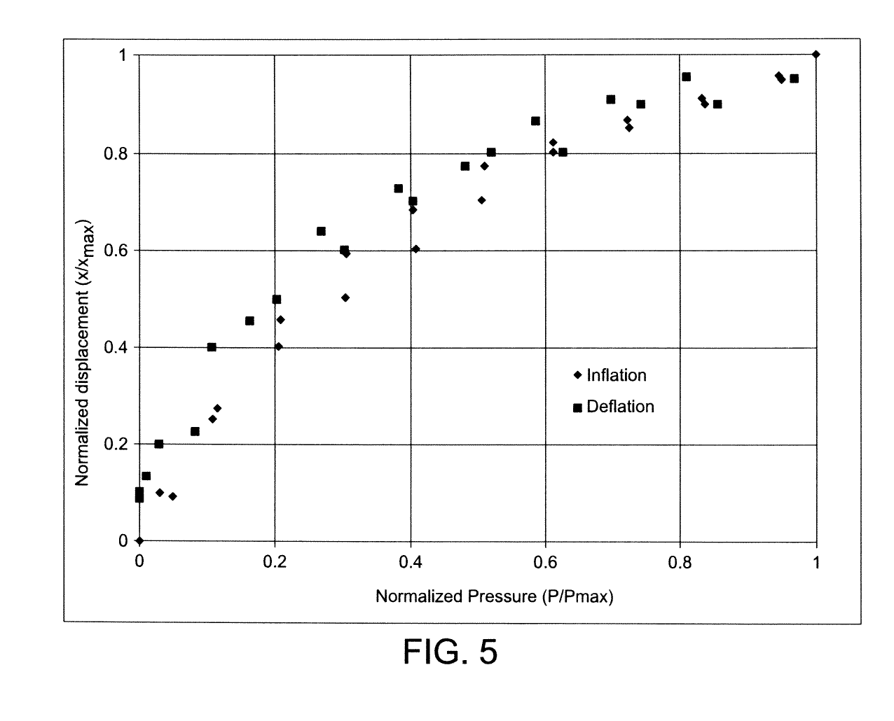

The indicator embodiment shown in FIG. 3 was constructed and tested. The indicator balloon was molded to an approximately spherical shape of 1.25 cm diameter from polyurethane tubing. The needle for this indicator was a flexible Tygon® tube (OD 0.2 cm) assembled in a fixed-to-the-wall configuration with a 2 cm cantilever length. The indicator was connected in series with a MicroCuff® endotracheal tube and a Dwyer digital pressure gage. The ET tube was inflated and deflated while simultaneously monitoring the cuff pressure with the pressure gage and the recording the tip displacement of the indicator. The pressure in the cuff is normalized with a maximum pressure that corresponds to when the need tip displacement reached a maximum value. The tip displacement was normalized with this maximum displacement value. The results from this are shown in FIG. 5, showing good displacement sensitivity to cuff pressure across the entire range of measurement. The squares in FIG. 5 denote deflation and the diamonds denote inflation.

Other arrangements are included in the spirit and scope of the disclosure. As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A pressure indicator for a tracheal catheter with an inflatable cuff, said pressure indicator comprising a balloon and needle wherein said balloon inflates and deflates in response to changes in pressure in said cuff and said needle moves in response to said inflation and deflation of said balloon.

2. The cuffed tracheal catheter pressure indicator of claim 1 wherein said balloon is in fluid communication with said cuff and said needle is in mechanical communication with said balloon.

3. The cuffed tracheal catheter pressure indicator of claim 1 wherein said balloon is made from a thin material so that it can respond quickly and with minimal error to changes in the cuff pressure.

4. The cuffed tracheal catheter pressure indicator of claim 3 wherein said balloon has a thickness of from 1 micron to 25 microns.

5. The cuffed tracheal catheter pressure indicator of claim 1 wherein said balloon is made from a material selected from the group consisting of polyethylene teraphihalate (PET), low-density polyethylene (LDPE), polyvinyl chloride (PVC), polyurethane (PU), polyolefin or polydimethylsiloxane (PDMS) polymers.

6. The cuffed tracheal catheter pressure indicator of claim 1 wherein said indicator has a range of from 0 to 70 cmH₂O.

7. The cuffed tracheal catheter pressure indicator of claim 1 wherein said indicator has a range of from 15 to 45 cmH₂O.

8. The cuffed tracheal catheter pressure indicator of claim 1 wherein said needle is fixedly attached to a frame.

9. The cuffed tracheal catheter pressure indicator of claim 1 wherein said needle is hingedly attached to a frame.

10. The cuffed tracheal catheter pressure indicator of claim 9 further comprising a restorative force to move said needle back towards its starting point as the pressure in said balloon decreases.

11. A tracheal catheter comprising a cannula having at least a respiratory lumen and an inflation lumen, said inflation lumen terminating in an inflatable cuff on a distal end, said inflation lumen having the pressure indicator of claim 1 on a proximal end.

12. The tracheal catheter of claim 11, wherein said balloon is in fluid communication with said cuff and said needle is in mechanical communication with said balloon.
